(19) United States
(12) Patent Application Publication
(10) Pub. No.: US 2024/0378346 A1
(43) Pub. Date: Nov. 14, 2024

(54) THIN-WALLED SHELL MODEL UPDATING METHOD AND SYSTEM

(71) Applicant: Dalian University of Technology, Dalian (CN)

(72) Inventors: Xiangtao MA, Dalian (CN); Bo WANG, Dalian (CN); Peng HAO, Dalian (CN); Zitong ZHOU, Dalian (CN)

(73) Assignee: Dalian University of Technology, Dalian (CN)

(21) Appl. No.: 18/784,658

(22) Filed: Jul. 25, 2024

Related U.S. Application Data
(63) Continuation of application No. PCT/CN2022/098048, filed on Jun. 10, 2022.

(30) Foreign Application Priority Data
Jan. 25, 2022 (CN) 202210098958.3

(51) Int. Cl. G06F 30/23; G06F 30/15

(52) U.S. Cl. CPC G06F 30/23; G06F 30/15

(57) ABSTRACT

The present application provides a thin-walled shell model updating method and system. The method includes converting an influence of fine feature structures in a space shell structure on stiffness and mass equivalently onto a skin and a stiffener, so as to simulate the influence of the fine feature structures on a bearing capacity of a whole structure.

### CROSS-REFERENCE TO RELATED APPLICATIONS

This application is a continuation application of International Application No.: PCT/CN2022/098048, filed on Jun. 10, 2022, which is based upon and claims priority to Chinese Patent Application No. 202210098958.3, filed on Jan. 25, 2022, the entire disclosures of which are incorporated by reference for all purposes.

### FIELD

The present application belongs to the technical field of analysis and design of main bearing members of aerospace structures, and particularly relates to a thin-walled shell model updating method and system.

### BACKGROUND

Because of high specific stiffness and high specific strength, thin-walled shell structures are widely used in main bearing members of space equipment such as launch vehicles. During a rocket launch, the thin-walled shell structure is prone to buckling instability due to huge axial compression load because of overload, thereby leading to structural failure. Because of design characteristics, the space equipment usually pursues ultimate weight reduction; numerical simulation techniques such as finite element analysis are needed for multiple analyses in a design process so as to predict the actual bearing capacity of a thin-walled shell under an axial compression condition and to realize the optimization iteration of the structure; and therefore, the finite element analysis on the bearing capacity of the thin-walled shell with high fidelity is an important premise of the lightweight design of the main bearing thin-walled structure of the space equipment such as the launch vehicles.

However, due to the manufacturing processes such as milling, casting, welding and the like used in the space shell structures, there are often a large number of fine feature structures such as chamfers, welds, openings, rivets and flanges in the thin-walled shell structures, which cause great difficulties in the construction of simulation models. Taking the chamfer feature as an example, the traditional shell structure is often simulated by shell models or solid models, but the modeling with the shell model cannot consider the chamfer structure feature; and therefore, the structural stiffness and mass simulated by the shell model are lower than the real structure, and errors caused by the chamfer structure cannot be quantified, so that the structural design can be ensured to be available only by conservative design, which is easy to bring about structural overweight. The structural features such as chamfers can be reflected sufficiently by adopting the solid model, but in order to ensure the analysis precision, the solid model needs to be divided into multiple units along a skin and stiffener directions to avoid the misalignment problems of the model such as shear self-locking, which finally leads to large size of the model; particularly for large-diameter grid-stiffened shells, the degree of freedom of the solid model can reach hundreds of millions, and even billions; and moreover, the space shell structure needs time-consuming simulation algorithms such as post-buckling analysis, so that the calculation cost using the solid model for analysis is unacceptable. The above problems lead to that the analysis efficiency and accuracy of thin-walled shell structure cannot be balanced. Therefore, it is urgent to develop a thin-walled shell model updating method capable of considering the fine feature structures such as chamfers to realize high fidelity and efficient analysis of the complex space thin-walled shell structures.

### SUMMARY

According to a first aspect of the present application, some embodiments provide a thin-walled shell model updating method, including converting an influence of fine feature structures in a space shell structure on stiffness and mass equivalently onto a skin and a stiffener, so as to simulate the influence of the fine feature structures on a bearing capacity of a whole structure.

According to a second aspect of the present application, some embodiments provide a thin-walled shell model updating system, which includes: one or more processors; a non-transitory storage coupled to the one or more processors; and a plurality of programs stored in the non-transitory storage that, when executed by the one or more processors, cause the thin-walled shell model updating system to perform acts including: converting an influence of fine feature structures in a space shell structure on stiffness and mass equivalently onto a skin and a stiffener, so as to simulate the influence of the fine feature structures on a bearing capacity of a whole structure.

### BRIEF DESCRIPTION OF THE DRAWINGS

A more particular description of the examples of the present disclosure will be rendered by reference to specific examples illustrated in the appended drawings. Given that these drawings depict only some examples and are not therefore considered to be limiting in scope, the examples will be described and explained with additional specificity and details through the use of the accompanying drawings.

FIG. 1 is a flow chart of a method provided by an embodiment of the present application.

FIG. 2 is a schematic diagram of a local solid model and updating process in a method provided by an embodiment of the present application.

FIG. 3 is a structural schematic diagram of a model obtained by a method provided by an embodiment of the present application.

FIG. 4 illustrates a load-displacement curve of a method provided by an embodiment of the present application, a traditional method, and an experiment result.

### DETAILED DESCRIPTION

Reference will now be made in detail to specific implementations, examples of which are illustrated in the accompanying drawings. In the following detailed description, numerous non-limiting specific details are set forth in order to assist in understanding the subject matter presented herein. But it will be apparent to one of ordinary skill in the art that various alternatives may be used. For example, it will be apparent to one of ordinary skill in the art that the subject matter presented herein can be implemented on many types of electronic devices with digital video capabilities.

Reference throughout this specification to “one embodiment,” “an embodiment,” “an example,” “some embodiments,” “some examples,” or similar language means that a particular feature, structure, or characteristic described is included in at least one embodiment or example. Features, structures, elements, or characteristics described in connection with one or some embodiments are also applicable to other embodiments, unless expressly specified otherwise.

Throughout the disclosure, the terms “first,” “second,” “third,” and etc. are all used as nomenclature only for references to relevant elements, e.g., devices, components, compositions, steps, and etc., without implying any spatial or chronological orders, unless expressly specified otherwise. For example, a “first device” and a “second device” may refer to two separately formed devices, or two parts, components or operational states of a same device, and may be named arbitrarily.

The terms “module,” “sub-module,” “circuit,” “sub-circuit,” “circuitry,” “sub-circuitry,” “unit,” or “sub-unit” may include memory (shared, dedicated, or group) that stores code or instructions that can be executed by one or more processors. A module may include one or more circuits with or without stored code or instructions. The module or circuit may include one or more components that are directly or indirectly connected. These components may or may not be physically attached to, or located adjacent to, one another.

As used herein, the term “if” or “when” may be understood to mean “upon” or “in response to” depending on the context. These terms, if appear in a claim, may not indicate that the relevant limitations or features are conditional or optional. For example, a method may comprise steps of: i) when or if condition X is present, function or action X′ is performed, and ii) when or if condition Y is present, function or action Y′ is performed. The method may be implemented with both the capability of performing function or action X′, and the capability of performing function or action Y′. Thus, the functions X′ and Y′ may both be performed, at different times, on multiple executions of the method.

A unit or module may be implemented purely by software, purely by hardware, or by a combination of hardware and software. In a pure software implementation, for example, the unit or module may include functionally related code blocks or software components, that are directly or indirectly linked together, so as to perform a particular function.

An objective of the present application is to solve the difficulties in the prior art, and provide a thin-walled shell model updating method and system to solve the problems of analysis efficiency of a solid unit and analysis precision of a shell unit of a space shell structure, and realize the high fidelity and efficient finite element analysis of the thin-walled shell structure containing structural detail features such as chamfers, which establishes a connection between a thin-walled shell model and a solid model by using a homogenization method, and ensures the advantage of the calculation efficiency of the shell model and simulates the influence of the structural detail features such as the complex chamfers on the bearing capacity of the whole structure by equivalently converting the contribution of the structural detail features such as the chamfers to stiffness and mass onto skin and stiffeners.

Grid-stiffened cylindrical shells are usually used in launch vehicles, and are prone to buckling under an axial compression load and extremely sensitive to structural defects. Establishing a simulation model with high fidelity is a premise of correctly analyzing buckling load and defect sensitivity of the space shell structures.

However, due to factors such as process manufacturing, there are often a large number of fine feature structures such as chamfers, welds, openings and the like in the thin-walled shell structure, which leads to low analysis efficiency of the existing solid simulation model, and low analysis precision of the existing shell simulation model. In order to realize the efficient and high-fidelity simulation analysis of the space shell structure, the present application provides a thin-walled shell model updating method considering structural detail features such as chamfers, which improves the analysis precision of the model on the premise of ensuring the analysis efficiency of the structure, and meets the requirement on complex mechanical analysis such as buckling process, defect sensitivity and the like.

The present application provides a thin-walled shell model updating method, which converts the influence of fine feature structures in a space shell structure on stiffness and mass equivalently onto skin and stiffeners so as to simulate the influence of the fine feature structure on the bearing capacity of the whole structure.

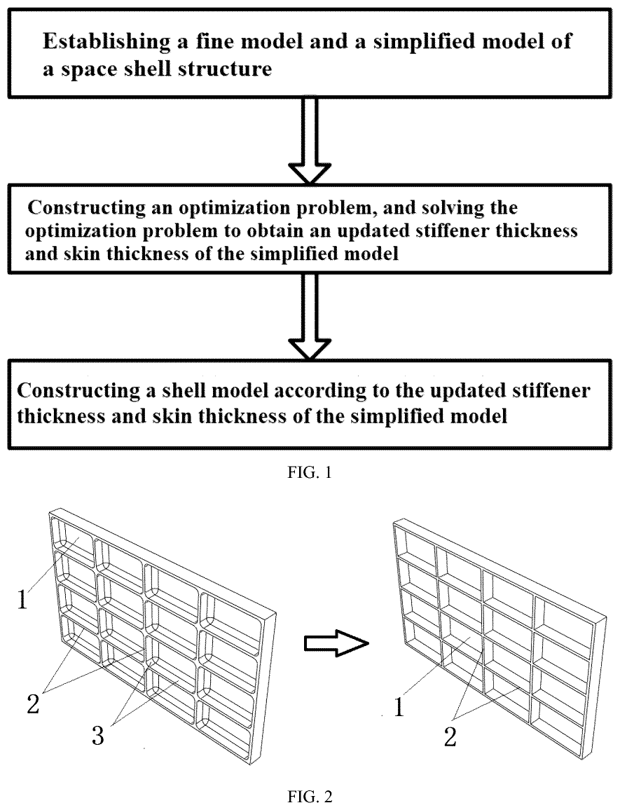

Specifically, as shown in FIG. 1, the method of the present application includes the following steps:

Step I, establishing a fine model and a simplified model of a space shell structure:

As shown in FIG. 2, firstly, establishing a local refined solid model containing a fine feature structure of a space shell structure, i.e. a fine model Ω₀ (which is modeled by using the existing commercial finite element software such as ABAQUS, ANSYS and the like, and is not repeated here.), wherein the fine model Ω₀ includes skin 1, stiffeners 2 and fine feature structures 3, wherein a stiffener height of the fine model Ω₀ is h_{stiffener}, and a stiffener thickness and a skin thickness are t_{stiffener} and tₛₖᵢₙ respectively. Equivalent elastic modulus constants E⁰₁, E⁰₂, E⁰₃ of the fine model Ω₀ are obtained by a homogenization method, which are equivalent elastic modulus constants in three axial directions X, Y and Z respectively.

Then, establishing a local refined solid model without a fine feature structure of the space shell structure, i.e. a simplified model Ω₁ (which is modeled by using the existing commercial finite element software such as ABAQUS, ANSYS and the like, and is not repeated here.), wherein the simplified model Ω₁ includes skin 1 and stiffeners 2, wherein a stiffener height of the simplified model Ω₁ is h_{stiffener}, and a stiffener thickness and a skin thickness are t_{stiffener}+Δt_{stiffener} and tₛₖᵢₙ+Δtₛₖᵢₙ respectively. Equivalent elastic modulus constants E₁, E₂, E₃ of the simplified model Ω₁ are obtained by a homogenization method, which are equivalent elastic modulus constants in three axial directions X, Y and Z respectively.

The homogenization method is an existing technology for equating structural feature cells to an anisotropic material and calculating the equivalent elastic modulus constant, and a specific technological process is briefed as follows:
(1) a periodic displacement boundary condition is attached to the local solid model to successively obtain a unit feature strain and corresponding stress.

$${\overline{σ}}_{ij} = \frac{{( {P}_{i} )}_{j}}{{S}_{j}}$$

$$( i , j = 1 , 2 , 3 )$$

In the formula, (Pᵢ)ⱼ indicates a counterforce in a direction i of a j^{th} outer surface, and Sⱼ indicates an area of the j^{th} outer surface. Then an overall average stress and average strain of the local solid model are obtained according to the following formula.

$${\overline{σ}}_{ij} = \frac{1}{V} {\int }_{V} {σ}_{ij} dV ,$$

$${\overline{ε}}_{ij} = \frac{1}{V} {\int }_{V} {ε}_{ij} dV ,$$

$$( i , j = 1 , 2 , 3 )$$

(2) The equivalent elastic modulus constant of the local solid model is calculated according to the following formula.

$${E}_{i} = \frac{{\overline{σ}}_{i}}{{\overline{ε}}_{i}} ,$$

$${μ}_{ij} = - \frac{{\overline{ε}}_{i}}{{\overline{ε}}_{j}} ,$$

$${G}_{ij} = \frac{{\overline{σ}}_{ij}}{{\overline{ε}}_{ij}} ,$$

$$( i , j = 1 , 2 , 3 )$$

In some embodiments, the above homogenization method may be implemented in various ways, including a representative volume element method, an asymptotic homogenization method and the like.

Further, the local refined solid model mentioned in step I includes various models, such as smallest representative cells, multiple cells, etc., which are all existing models, and only one of them can be selected when the present application is used.

Further, the fine feature structure mentioned in step I includes chamfers (round chamfers and square chamfers), or welds, openings, rivets, flanges and fine feature structures caused by other processes, the welds, openings, rivets and flanges are structural features at the same level as chamfers, and the fine feature structure in the embodiment shown in FIG. 1 is the chamfer.

Step II, constructing an optimization problem, and solving the optimization problem to obtain an updated stiffener thickness and skin thickness of a simplified model:

There are two optimization variables in the optimization problem, i.e. a difference Δt_{stiffener} between the stiffener thickness of the simplified model Ω₁ and the stiffener thickness of the fine model Ω₀ and a difference Δtₛₖᵢₙ between the skin thickness of the simplified model Ω₁ and the skin thickness of the fine model Ω₀.

An optimization target is to minimize an equivalent elastic modulus constant difference ∥(Eᵢ−E⁰ᵢ)∥₂ between the solid model Ω₁ without fine chamfer features and the local refined solid model Ω₀ containing chamfers (the target here is 2 norms of the difference between two vectors (a length is 3), that is, to achieve the minimum difference between the three elastic modulus constants), to obtain updated skin and stiffener sizes, that is, to find Δt_{stiffener} and Δtₛₖᵢₙ that minimize the difference of the three constants, i.e. the optimal Δt_{stiffener} and Δtₛₖᵢₙ, and then to obtain the updated stiffener thickness and skin thickness of the simplified model according to t_{stiffener}+Δt_{stiffener} and tₛₖᵢₙ+Δtₛₖᵢₙ.

A formula of the optimization problem is as follows:

$$min f ( Δ {t}_{stiffener} , Δ {T}_{skin} ) = {‖ ( {E}_{i} - {E}_{i}^{0} ) ‖}_{2} ,$$

$$i = 1 , 2 , 3$$

$$subject to :$$

$$\underline{Δ {t}_{stiffener}} < Δ {t}_{stiffener} < \overline{Δ {t}_{stiffener}}$$

$$\underline{Δ {t}_{skin}} < Δ {t}_{skin} < \overline{Δ {t}_{skin}}$$

An upper line in the above formula indicates an upper limit value of a variable, a lower line indicates a lower limit value of the variable, the upper limit value and the lower limit value are given manually, and it may be suggested that the upper limit value and the lower limit value are set as 150% and 50% of an initial size respectively.

The above formula indicates that the variables are adjustment values Δt_{stiffener} and Δtₛₖᵢₙ of the stiffener thickness and the skin thickness respectively, which aims at minimizing an error of the equivalent elastic constant of the simplified model and the fine model, and the lower limit and upper limit of constants are the lower limit value and upper limit value of an adjustment size.

In the above process, the equivalent elastic attributes of the two models are calculated respectively by using a homogenization method, and size parameters of the simplified model are adjusted by an optimization algorithm, so that features of the simplified model are the same as the features of the fine model, which simulates the influence of the fine feature structures such as complex chamfers on the bearing capacity of the whole structure.

Further, a method of solving the optimization problem mentioned in step II may adopt a genetic algorithm, an ant colony algorithm, a particle swarm optimization or sequential quadratic programming and other optimization algorithms and optimization strategies such as surrogate model optimization, continuous distributed optimization or hybrid optimization.

Step III, constructing a shell model according to the updated stiffener thickness and skin thickness of the simplified model, and performing simulation analysis according to the requirement of working conditions.

The same software as in step I is used for constructing the shell model according to the updated stiffener thickness and skin thickness of the simplified model, and the modeling in step III differs from that in step I in that the solid modeling is used in step I, and shell modeling is used in step III.

The shell model used in step III in the present application is a finite element model, and the calculation cost of the shell model is low, so that a calculation model with high efficiency and high fidelity is obtained, and the advantage of the calculation efficiency of the shell model is fully used.

An implementation of the method of the present application is as follows:

Axial compression experiment and high-precision simulation of a grid-stiffened space shell

In order to study the defect sensitivity and buckling behavior of the grid-stiffened shell, a square grid-stiffened shell with a diameter of 1.6 m and a height of 1.0 m is manufactured by using an integral manufacturing technology, which is used for the axial compression buckling test; and high precision and few initial defects are ensured by casting and numerical control milling technology.

Stiffeners of the grid-stiffened shell are distributed densely and uniformly. Specific size measurement is as follows: the number of circumferential stiffeners is N_{c}=17, the number of axial stiffeners is N_{A}=138 (that is, there are 17 annular stiffeners, and 138 stiffeners parallel to the central axis), a thickness is tᵣ=2.5 mm, a height is h=11.9 mm, and a skin thickness is tₛ=1.6 mm. There is a chamfer with a radius of 4 mm between the skin and the stiffener. The structural material is aluminum alloy with the elastic modulus constant of E=76169 MPa and a poisson ratio of v=0.3. A dual-linear elastic model is used, a buckling limit is 339.58 MPa, a strength limit is 437.92 MPa, and an elongation rate is 12.92%. Performance parameters of the above material are obtained through material test.

An explicit dynamic analysis method considering geometric nonlinearity and material nonlinearity is used in simulation analysis to simulate the buckling behavior of the structure. The bottom end of the model is fixed, and a homogeneous displacement boundary is applied to the upper end to simulate the displacement control of the experiment. Two units are distributed in the height direction of the stiffener, the size of the whole grid is set to be 5.95 mm, the whole grid is divided into 98256 units, Abaqus software is used for analysis, and S4R is adopted as the unit type.

Step I, establishing models Ω₀ and Ω₁ (the models here are the local cell model of the aforementioned entity or a local model formed by several cells), wherein the model is regarded as a cell in the homogenization method, and a stiffening plate of each cell includes five peripheral stiffeners and five axial stiffeners. Elastic modulus constants E⁰₁, E⁰₂, E⁰₃ of the fine model Ω₀ are 8481.9 Mpa, 7447.8 MPa and 6241.8 Mpa respectively.

Step II, establishing the optimization of model updating, wherein the optimization target is to minimize the difference of the equivalent elastic modulus constants, and optimization variables are differences between the stiffener thickness and skin thickness of the simplified model and the stiffener thickness and skin thickness of the fine model: Δt_{stiffener} and Δtₛₖᵢₙ. In the present embodiment, SQP-NLPQL based on gradient is used as the optimization algorithm. Optimization results are shown in Table 1.

TABLE 1

| | | | | Mass | | | | |
|---|---|---|---|---|---|---|---|---|
| | tₛₖᵢₙ | t_{stiffener} | Mass | error | E₁ | E₂ | E₃ | |
| | [mm] | [mm] | [kg] | [%] | [MPa] | [MPa] | [MPa] | [MPa] |
| Entity model with | 1.60 | 2.50 | 0.348 | — | 8481.9 | 7447.8 | 6241.8 | — |
| chamfer angle | | | | | | | | |
| Solid model without a | 1.60 | 2.50 | 0.315 | −9.48 | 7670.9 | 6656.5 | 5429.8 | 1454.4 |
| chamfer | | | | | | | | |
| Method model of the | 1.76 | 2.87 | 0.353 | 1.44 | 8590.8 | 7439.2 | 6237.2 | 13.25 |
| present application | | | | | | | | |

The “method model of the present application” in Table 1 refers to the shell model constructed by using the updated stiffener thickness and skin thickness of the simplified model, and the shell model is shown in FIG. 3-1. The solid model with the chamfer in Table 1 is the fine model Ω₀, and the solid model without the chamfer is the simplified model Ω₁.

It may be seen from the results of Table 1 that for the present embodiment, the chamfer accounts for about 10% of the overall mass of the structure. Therefore, it is necessary to consider the influence of the chamfer on the structure in a modeling process. The mass and equivalent elastic modulus constant of the shell model constructed in step II are kept consistent with the fine model Ω₀ with the chamfers. Furthermore, the bearing capacity of the perfect geometric shell model (i.e. the model without any defect) after the size updated by the method of the present application is 4537.61 kN (calculated by using the finite element software for axial compression buckling analysis, and the bearing capacity is obtained by using the explicit dynamic analysis algorithm in ABAQUS software in the present embodiment), which is 12.23% more than the bearing capacity of the shell model obtained by the traditional method.

Step III, performing an axial compression experiment for the shell by using a stiffened shell axial compression test platform

The stiffened shell axial compression test platform is the existing experimental apparatus, which is not repeated here. Before the axial compression test for the shell, three lateral disturbing loads are uniformly applied to a middle position of the height of a side wall of the shell along a circumferential direction, and three initial pit geometric defects (i.e. No. 1 defect to No. 3 defect) are prefabricated, and the initial geometric defects are shown in Table 2.

TABLE 2

| | No. 1 defect | No. 2 defect | No. 3 defect |
|---|---|---|---|
| Disturbing load amplitude | 9.71 | 9.75 | 9.80 |
| [kN] | | | |
| Defect amplitude [mm] | 1.959-2.259 | 1.898-2.198 | 1.767-2.067 |

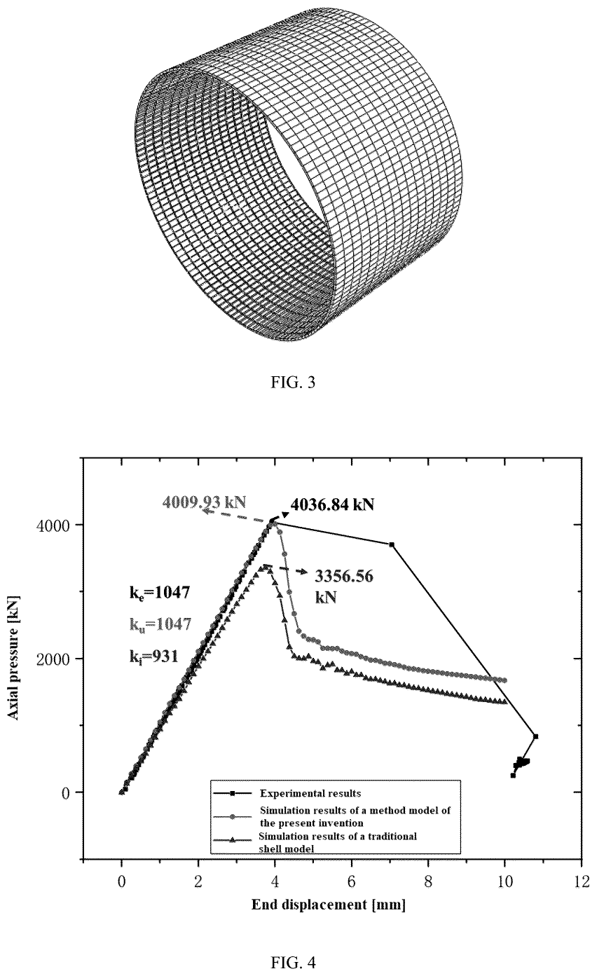

For comparison and validation, the above three defects are added to the perfect geometric shell model constructed by the method of the present application and the shell model constructed by the traditional method, and load experiments are performed on the entity with the defects, the shell model constructed by the traditional method and the shell model constructed by the method of the present application; load-displacement curves of the method of the present application, the traditional method and the experimental results are shown in FIG. 4 (the “experimental results” in FIG. 4 are obtained by loading the entity with the three defects using the above stiffened shell axial compression test platform; “the method model of the present application” and “the traditional shell model” are modeled and analyzed for the same entity, and the models are added with the three defects; and the results of the two models are obtained by using the finite element software such as ABAQUS.

It can be found from FIG. 4 that the bearing capacity obtained by the experiment on the entity shell is 4036.84 kN, the bearing capacity of the traditional shell model is 3356.56 kN, and the bearing capacity of the method model of the present application is 4009.93 kN; and therefore, an analysis error of the traditional shell model is 16.85%, and the analysis error of the method model of the present application is only 0.67%. It may be seen from the above analysis that compared with the experimental result of the entity, the finite element model updated by the method of the present application has higher analysis precision than the traditional shell finite element model without considering the influence of the fine feature structure such as the chamfer.

In conclusion, the thin-walled shell updating method considering the fine feature structure such as the chamfer provided by the present application can provide the high-efficient and high-fidelity simulation model, which can precisely simulate complex mechanical properties such as the buckling process and defect sensitivity analysis.

Some embodiments of the present application further provide a thin-walled shell model updating system, which includes:
a solid model establishing unit used for establishing a fine model and a simplified model of a space shell structure;
an optimization problem solving unit connected with the solid model establishing unit, and used for constructing an optimization problem, and solving the optimization problem to obtain an updated stiffener thickness and skin thickness of the simplified model; and
a shell model establishing unit connected with the optimization problem solving unit and used for constructing a shell model according to the updated stiffener thickness and skin thickness of the simplified model.

Compared with the prior art, the present application has the following beneficial effects:

In the design of space shells, the influence of the fine feature structures such as the chamfers on the structural mass and stiffness is fully considered in the present application, and the simulation model is updated through the optimization algorithm based on homogenization, so that the structural behavior, local buckling behavior and post-buckling behavior of a grid-stiffened cylinder when the material exceeds a yield limit and enters a plastic stage can be accurately simulated. The present application has both the analysis precision of the solid model method and the analysis efficiency of the shell model, solves the problems of high calculation cost caused by the refining and lightweight design of the complex thin-walled structure in the launch vehicles, and can provide design methods, optimization tools and guidance for the design of the main bearing shell structures in the new generation of launch vehicles.

In some embodiments, the method includes:
step I, establishing a fine model and a simplified model of a space shell structure;
step II, constructing an optimization problem, and solving the optimization problem to obtain an updated stiffener thickness and skin thickness of the simplified model; and
step III, constructing a shell model according to the updated stiffener thickness and skin thickness of the simplified model.

In some embodiments, an operation of the step I includes:

Firstly, establishing a local refined solid model containing a fine feature structure of the space shell structure, i.e. a fine model Ω₀, wherein the fine model Ω₀ includes skin, stiffeners and fine feature structures; the stiffener thickness and skin thickness of the fine model Ω₀ are t_{stiffener} and tₛₖᵢₙ respectively; and obtaining equivalent clastic modulus constants E⁰₁, E⁰₂, E⁰₃ of the fine model Ω₀ by using a homogenization method;

Then, establishing a local refined solid model without the fine feature structure of the space shell structure, i.e. a simplified model Ω₁, wherein the simplified model Ω₁ includes skin and stiffeners; the stiffener thickness and skin thickness of the simplified model Ω₁ are t_{stiffener}+Δt_{stiffener} and tₛₖᵢₙ+Δtₛₖᵢₙ respectively; and obtaining equivalent elastic modulus constants E₁, E₂, E₃ of the simplified model Ω₁ by using a homogenization method.

In some embodiments, the homogenization method adopts a representative volume element method or an asymptotic homogenization method.

In some embodiments, the local refined solid model includes a minimal representative cell model or a plurality of cell models.

In some embodiments, the fine feature structure includes chamfers, welds, openings, rivets or flanges.

In some embodiments, a formula of the optimization problem constructed in step II is as follows:

$$min f ( Δ {t}_{stiffener} , Δ {T}_{skin} ) = {‖ ( {E}_{i} - {E}_{i}^{0} ) ‖}_{2} ,$$

$$i = 1 , 2 , 3$$

$$subject to :$$

$$\underline{Δ {t}_{stiffener}} < Δ {t}_{stiffener} < \overline{Δ {t}_{stiffener}}$$

$$\underline{Δ {t}_{skin}} < Δ {t}_{skin} < \overline{Δ {t}_{skin}}$$

An upper line in the above formula indicates an upper limit value of a variable, and a lower line indicates a lower limit value of the variable.

In some embodiments, in the step II, an operation of solving the optimization problem to obtain the updated stiffener thickness and skin thickness of the simplified model includes:
solving the optimization problem to obtain an optimal Δt_{stiffener} and Δtₛₖᵢₙ, and then obtaining the updated stiffener thickness and skin thickness of the simplified model according to t_{stiffener}+Δt_{stiffener} and tₛₖᵢₙ+Δtₛₖᵢₙ.

In some embodiments, the optimization algorithm used for solving the optimization problem in step II includes a genetic algorithm, an ant colony algorithm, particle swarm optimization or sequential quadratic programming; and used optimization strategies include surrogate model optimization, continuous distributed optimization or hybrid optimization.

In some embodiments of the present application, the thin-walled shell model updating system may be a computational system comprising one or more processors. The one or more processors may execute instructions to perform all or some of the steps in the above-described methods. The instructions may be stored in a non-transitory computer-readable storage medium coupled to the one or more processors. Moreover, the one or more processors may include one or more modules that facilitate the interaction between the processor and other components. The processor may be a Central Processing Unit (CPU), a microprocessor, a single chip machine, a Graphical Processing Unit (GPU), or the like.

The above embodiments are merely exemplary implementations of the present application. Based on the application method and principle disclosed by the present application, various improvements or modifications may be provided by other embodiments, which are not limited to the method described in the embodiments of the present application; and therefore, the aforementioned way is only preferred, and has no restrictive significance.

What is claimed is:

1. A thin-walled shell model updating method, comprising:
converting an influence of fine feature structures in a space shell structure on stiffness and mass equivalently onto a skin and a stiffener, so as to simulate the influence of the fine feature structures on a bearing capacity of a whole structure.

2. The thin-walled shell model updating method according to claim 1, wherein converting the influence of fine feature structures in the space shell structure on stiffness and mass equivalently onto the skin and the stiffener comprises:
establishing a fine model and a simplified model of the space shell structure;
constructing an optimization problem, and solving the optimization problem to obtain an updated stiffener thickness and an updated skin thickness of the simplified model; and
constructing a shell model according to the updated stiffener thickness and the updated skin thickness of the simplified model.

3. The thin-walled shell model updating method according to claim 2, wherein establishing the fine model and the simplified model of the space shell structure comprises:
establishing a first local refined solid model comprising a fine feature structure of the space shell structure, wherein the first local refined solid model comprises the fine model Ω₀, the fine model Ω₀ comprises the skin, the stiffener and the fine feature structures, a stiffener thickness and a skin thickness of the fine model Ω₀ are t_{stiffener} and tₛₖᵢₙ respectively, and equivalent elastic modulus constants E⁰₁, E⁰₂, E⁰₃ of the fine model Ω₀ are obtained by using a homogenization method;
establishing a second local refined solid model without the fine feature structure of the space shell structure, wherein the second local refined solid model comprises a simplified model Ω₁, the simplified model Ω₁ comprises the skin and the stiffeners; a stiffener thickness and a skin thickness of the simplified model Ω₁ are t_{stiffener}+Δt_{stiffener} and tₛₖᵢₙ+Δtₛₖᵢₙ respectively, and equivalent elastic modulus constants E₁, E₂, E₃ of the simplified model Ω₁ are obtained by using a homogenization method.

4. The thin-walled shell model updating method according to claim 3, wherein the homogenization method adopts a representative volume element method or an asymptotic homogenization method.

5. The thin-walled shell model updating method according to claim 3, wherein the first local refined solid model or the second local refined solid model comprises a minimal representative cell model or a plurality of cell models.

6. The thin-walled shell model updating method according to claim 3, wherein the fine feature structure comprises chamfers, welds, openings, rivets, or flanges.

7. The thin-walled shell model updating method according to claim 3, wherein constructing the optimization problem comprises:
determining Δt_{stiffener} and Δtₛₖᵢₙ to minimize an equivalent elastic modulus constant difference ∥(Eᵢ−E⁰ᵢ)∥₂.

8. The thin-walled shell model updating method according to claim 7, wherein solving the optimization problem to obtain the updated stiffener thickness and the updated skin thickness of the simplified model comprises:
solving the optimization problem to obtain an optimal Δt_{stiffener} and Δtₛₖᵢₙ; and
obtaining the updated stiffener thickness and the updated skin thickness of the simplified model according to t_{stiffener}+Δt_{stiffener} and tₛₖᵢₙ+Δtₛₖᵢₙ.

9. The thin-walled shell model updating method according to claim 7, wherein an optimization algorithm used for solving the optimization problem comprises a genetic algorithm, an ant colony algorithm, particle swarm optimization or sequential quadratic programming, or
an optimization strategy used for solving the optimization problem comprises surrogate model optimization, continuous distributed optimization or hybrid optimization.

10. A thin-walled shell model updating system, comprising:
one or more processors;
a non-transitory storage coupled to the one or more processors; and
a plurality of programs stored in the non-transitory storage that, when executed by the one or more processors, cause the thin-walled shell model updating system to perform acts comprising:
converting an influence of fine feature structures in a space shell structure on stiffness and mass equivalently onto a skin and a stiffener, so as to simulate the influence of the fine feature structures on a bearing capacity of a whole structure.

11. The thin-walled shell model updating system according to claim 10, wherein converting the influence of fine feature structures in the space shell structure on stiffness and mass equivalently onto the skin and the stiffener comprises:
establishing a fine model and a simplified model of the space shell structure;
constructing an optimization problem, and solving the optimization problem to obtain an updated stiffener thickness and an updated skin thickness of the simplified model; and
constructing a shell model according to the updated stiffener thickness and the updated skin thickness of the simplified model.

12. The thin-walled shell model updating system according to claim 11, wherein establishing the fine model and the simplified model of the space shell structure comprises:
establishing a first local refined solid model comprising a fine feature structure of the space shell structure, wherein the first local refined solid model comprises the fine model Ω₀, the fine model Ω₀ comprises the skin, the stiffener and the fine feature structures, a stiffener thickness and a skin thickness of the fine model Ω₀ are t_{stiffener} and tₛₖᵢₙ respectively, and equivalent elastic modulus constants E⁰₁, E⁰₂, E⁰₃ of the fine model Ω₀ are obtained by using a homogenization method;
establishing a second local refined solid model without the fine feature structure of the space shell structure, wherein the second local refined solid model comprises a simplified model Ω₁, the simplified model Ω₁ comprises the skin and the stiffeners; a stiffener thickness and a skin thickness of the simplified model Ω₁ are t_{stiffener}+Δt_{stiffener} and tₛₖᵢₙ+Δtₛₖᵢₙ respectively, and equivalent elastic modulus constants E₁, E₂, E₃ of the simplified model Ω₁ are obtained by using a homogenization method.

13. The thin-walled shell model updating system according to claim 12, wherein the homogenization method adopts a representative volume element method or an asymptotic homogenization method.

14. The thin-walled shell model updating system according to claim 12, wherein the first local refined solid model or the second local refined solid model comprises a minimal representative cell model or a plurality of cell models.

15. The thin-walled shell model updating system according to claim 12, wherein the fine feature structure comprises chamfers, welds, openings, rivets, or flanges.

16. The thin-walled shell model updating system according to claim 12, wherein constructing the optimization problem comprises:
determining Δt_{stiffener} and Δtₛₖᵢₙ to minimize an equivalent elastic modulus constant difference ∥(Eᵢ−E⁰ᵢ)∥₂.

17. The thin-walled shell model updating system according to claim 16, wherein solving the optimization problem to obtain the updated stiffener thickness and the updated skin thickness of the simplified model comprises:
solving the optimization problem to obtain an optimal Δt_{stiffener} and Δtₛₖᵢₙ; and
obtaining the updated stiffener thickness and the updated skin thickness of the simplified model according to t_{stiffener}+Δt_{stiffener} and tₛₖᵢₙ+Δtₛₖᵢₙ.

18. The thin-walled shell model updating system according to claim 16, wherein an optimization algorithm used for solving the optimization problem comprises a genetic algorithm, an ant colony algorithm, particle swarm optimization or sequential quadratic programming, or
an optimization strategy used for solving the optimization problem comprises surrogate model optimization, continuous distributed optimization or hybrid optimization.
